(19) United States
(12) Patent Application Publication
(10) Pub. No.: US 2010/0192958 A1
(43) Pub. Date: Aug. 5, 2010

(54) PROPHYLACTIC DEVICE FOR USE DURING INTERCOURSE

(76) Inventors: Mark R. Cozens, Scottsdale, AZ (US); Alanda F. Cozens, Scottsdale, AZ (US)

Correspondence Address: Mark R. Cozens, 10081 East Desert Cove Avenue, Scottsdale, AZ, 85260, US

(21) Appl. No.: 12/700,622

(22) Filed: Feb. 4, 2010

Related U.S. Application Data
(60) Provisional application No. 61/149,776, filed on Feb. 4, 2009.

(51) Int. Cl. A61F 6/06

(52) U.S. Cl. 128/830

(57) ABSTRACT

Embodiments of the present invention may provide a prophylactic device comprises an elongated hollow tube including a shaft, a rounded closed end, and an open end that forms an elongated hollow cavity within the tube.

### CROSS-REFERENCE TO RELATED APPLICATIONS

This application claims the benefit of U.S. Provisional Patent Application No. 61/149,776, filed Feb. 4, 2009.

### BACKGROUND OF THE INVENTION

The present invention generally relates to a prophylactic device, and more specifically relates to a prophylactic device for use primarily by women during intercourse that may be worn much like underwear and that may help to prevent transmission of sexually transmitted diseases.

### SUMMARY OF THE INVENTION

In one aspect of the present invention, a prophylactic device may comprise an elongated hollow tube including a shaft, a rounded closed end, and an open end that forms an elongated hollow cavity within the tube.

In another aspect of the present invention, a prophylactic device may comprise an elongated hollow tube including a shaft, a rounded closed end, and an open end that forms an elongated hollow cavity within the tube, wherein the shaft of the tube maintains an elongated form regardless of whether an external object is inserted into the cavity of the tube; a flexible first triangular material having a first material end and a second material end, the second material end having first and second corners, is the flexible first triangular material being connected at the first material end to a first side of the open end of the tube, wherein the second material end is wider than the first material end; a flexible thin strip having a first strip end and a second strip end, the flexible thin strip being connected at the first strip end to a second side of the open end of the tube, wherein the second side of the open end of the tube is opposite from the first side of the open end of the tube; and a flexible second triangular material having a third material end and a fourth material end, the flexible second triangular material being connected at the third material end to the second strip end of the flexible thin strip, wherein the fourth material end is wider than the third material end.

These and other features, aspects and advantages of the present invention will become better understood with reference to the following drawings, description and claims.

### BRIEF DESCRIPTION OF THE DRAWINGS

FIG. 1 shows a perspective view of a prophylactic device in accordance with an embodiment of the present invention;

FIG. 2 shows a profile view of the prophylactic device of FIG. 1;

FIG. 3 shows an overhead view of the prophylactic device of FIG. 1;

FIG. 4 shows an underside view of the prophylactic device of FIG. 1;

FIG. 5 shows an interior profile view of the tube of the prophylactic device of FIG. 1;

FIG. 6 shows the prophylactic device of FIG. 1 attached to a belt in accordance with an embodiment of the present invention;

FIGS. 7a, 7b, and 7c show the prophylactic device of FIG. 1 attached to a user via the belt of FIG. 6 and inserted into the user's vaginal cavity; and

FIG. 8 shows an exploded perspective view of the prophylactic device of FIG. 1 and the belt of FIG. 6.

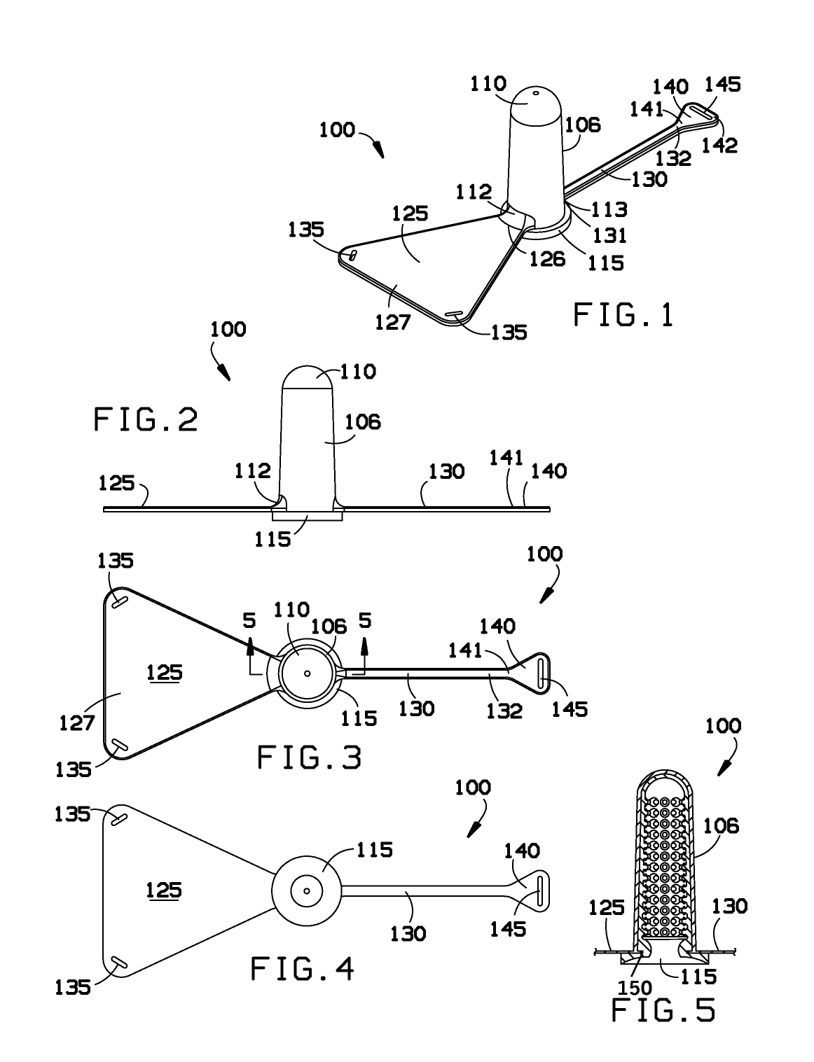

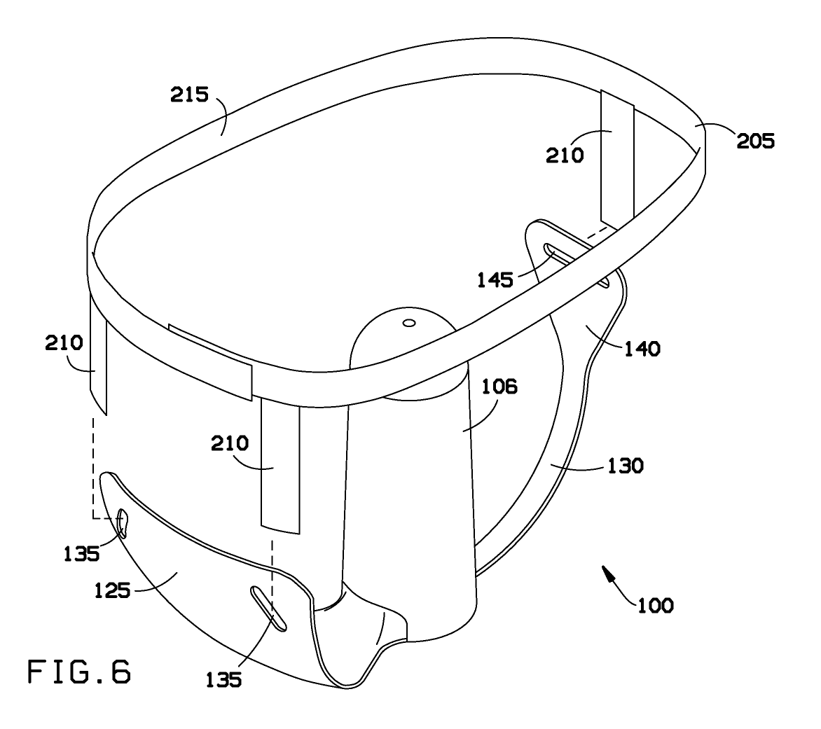

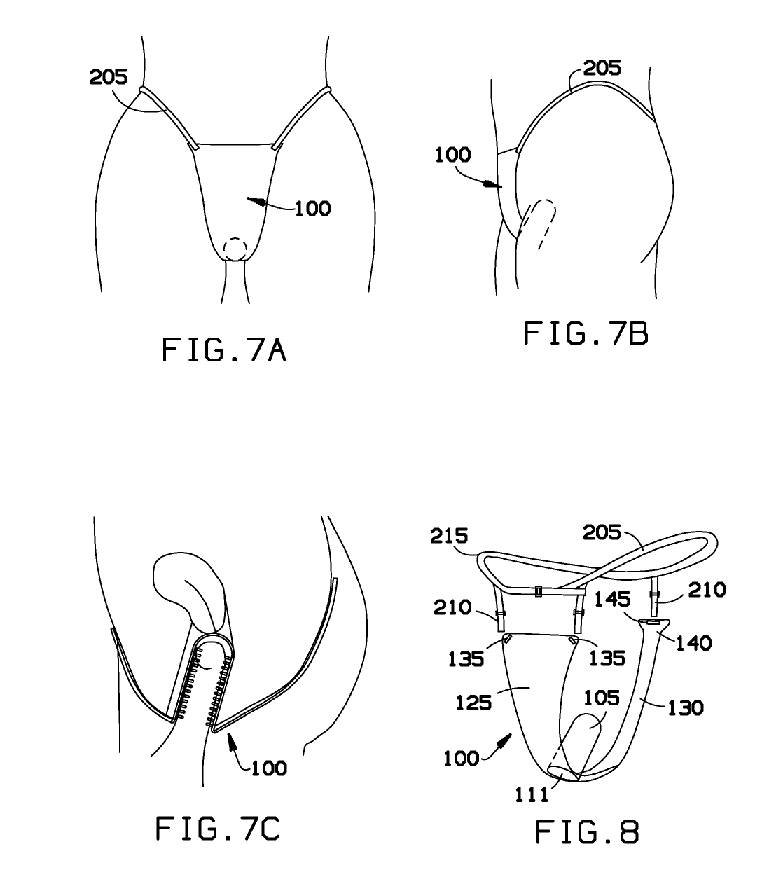

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a prophylactic device to be worn during sexual intercourse. The prophylactic device may be used primarily by women during intercourse and may be worn much like underwear. The device may also be durable and reusable, thus making the device more economical to use.

Referring now to the figures, an exemplary embodiment of the prophylactic device 100 may comprise an elongated hollow tube 105 including a shaft 106, a rounded closed end 110, and an open end 111 that forms an elongated hollow cavity within the interior of the tube 105. The tube 105 may be made of an elastomer, such as soft silicone rubber, having a low Young's modulus yet may have enough stiffness and/or rigidity to maintain an elongated form and not collapse regardless of whether an external object is inserted into the cavity of the tube 105. The tube 105 may also comprise an interior flanged lip 150 made of the same elastomer or a different material that may extend from and encircle an interior of the open end 111 that may help the tube 105 in maintaining its elongated form. In one exemplary embodiment, the tube 105 may be typically about 6 inches tall and about 2 inches in diameter, with a wall thickness of about 3/16 inch. The wall thickness may help to eliminate tearing of the tube and greater durability. A button component may be insertable into the open end 111 of the hollow tube 105 and may snap into the open end 111 and be secured to tube 105 at the open end 111 via the interior flanged lip. The button component may be operable to attach the tube 105 to any other materials by pressing against the tube and any other materials to be attached to the tube 105.

The tube 105 may also further comprise a plurality of columns of interior nubs 120 protruding perpendicularly from and lining an interior of the shaft 106 of the tube 105. Each of the plurality of columns of interior nubs 120 may run lengthwise along the interior of the shaft of the tube 105, and may be made of the same elastomer as the tube 105, or may be made of any other suitable material. The interior nubs 120 may be typically ¾ inches long spaced ⅛ inch apart, or may also be of any other length and/or spacing, and may also be made so that the tube 105 and the interior nubs 120 are of one piece, such as via molding or any other suitable process, or the interior nubs 120 may be separately attached to the tube 105 via adhesives or any other suitable means of attachment.

The prophylactic device may further comprise a flat and flexible first triangular material 125 having three corners, with one corner at a first end 126 of the first triangular material 125 connecting to and extending from a first side 112 of the open end 111 of the tube 105. The first triangular material 125 may be triangular in shape, so that the second end 127 of the first triangular material 125 opposite the first end 126 of the first triangular material 125 that is connected to the tube 105 is wider than the first end 126 of the first triangular material 125. The first triangular material 125 may also comprise two openings 135, such as slits, near the two corners opposite the first end 126 of the first triangular material 125. The first flexible material 125 may be made of any flexible material, such as the same elastomer as the tube 105, or may be made of any other suitable flexible material. The first flexible material 125 and the tube 105 may be made as a single piece of material, such as via molding, or the first flexible material 125 may be attached to the tube 105 via adhesives, via the button component via the button component pressing against the first flexible material 125 and the tube 105, or any other suitable way of attaching the first flexible material 125 to the tube 105.

The prophylactic device may also further comprise a flexible thin elongated strip 130 having first and second ends 131 and 132, with the first end 131 of the strip 130 connected to a second side 113 of the open end 111 of the tube 105 opposite from the first side 112 of the open end 111 of the tube 105. The second end 132 of the strip 130 may comprise or be connected to a flexible second triangular material 140 having first and second ends 141 and 142, with the first end 141 of the second triangular material 140 meeting the second end 132 of the strip 130 so that the second end 142 of the second triangular material 140 is wider than the first end 141 of the second triangular material 140. The second triangular material 140 may also comprise an opening 145, such as a slit, near its second end 142. The strip 130 and the flexible second triangular material 140 may be made of any flexible material, such as the same elastomer as the tube 105, or may be made of any other suitable flexible material. The strip 130, the flexible second triangular material 140, and the tube 105 may also be made as a single piece of material, such as via molding, or each of the components may be made individually and then be attached together via adhesives, via the button component via the button component pressing against the strip 130 and the tube 105, or any other suitable way of attaching the strip 130 to the tube 105.

In an exemplary embodiment of the present invention, a belt 205 may be attached to the prophylactic device 100 via attachment straps 210 on the belt 205, and the belt 205 may be secured to a user via a waist strap 215, so that the prophylactic device 200 may be secured to the user wearing the prophylactic device 200 and the belt 205. The attachment straps 210 on the belt 205 may be attached to the prophylactic device 200 through the two openings 135 on the first triangular material 125 and the opening 145 on the second triangular material 140.

In another exemplary embodiment of the present invention, the waist strap 215 of the belt 205 may be worn around the waist of a user, and the attachment straps 210 on the belt 205 may be attached to the prophylactic device 100. When worn, the belt 205 may cause the first triangular material 125 to be pulled up and over the front side of the user's crotch area and may cause the strip 130 and the second triangular material 140 to be pulled over the rear region of the user. Thus, the belt 205 may act to form the prophylactic much like the shape of thong underwear.

In use, a wearer, such as a female user, may insert the tube 105 of the prophylactic device 100 into her vaginal cavity 320. The waist strap 215 of the belt 305 may be worn around the waist of the user and the belt 205 may be attached to the prophylactic device 100 via the attachment straps 210 to the openings 135 and 145 on the prophylactic device 100 so that the prophylactic device 100 may be worn much like thong underwear by the user. In the act of sexual intercourse, an adult toy or a male sexual organ may be inserted into the tube 105 of the prophylactic device 100 through its opening 111 As the toy or sexual organ move within the tube of the prophylactic device 100, the internal nubs 120 may provide additional stimulation to the male sexual organ moving within the tube 105 of the prophylactic device 100. While the prophylactic device's 100 use is described in terms of a female user inserting the tube of the prophylactic device 100 into her vaginal cavity 320, the prophylactic device 100 may also be designed so that a wearer may insert the tube 105 into the anus of a user, and thus may be worn and used by both men and women.

It should be understood, of course, that the foregoing relates to exemplary embodiments of the invention and that modifications may be made without departing from the spirit and scope of the invention as set forth in the following claims.

We claim:

1. A prophylactic device comprising:
an elongated hollow tube including a shaft, a rounded closed end, and an open end that forms an elongated hollow cavity within the tube.

2. The device of claim 1, wherein the shaft of the tube maintains an elongated form regardless of whether an external object is inserted into the cavity of the tube.

3. The device of claim 2, further comprising:
an interior lip that extends from and encircles an interior of the second open end.

4. The device of claim 1, further comprising:
a plurality of columns of a plurality of interior nubs protruding from and lining an interior of the shaft.

5. The device of claim 1 further comprising:
a flexible first triangular material having a first material end and a second material end, the second material end of the flexible first triangular material having first and second corners, the flexible first triangular material being connected at the first material end to a first side of the open end, wherein the second material end of the flexible first triangular material is wider than the first material end of the flexible first triangular material;
a flexible thin strip having a first strip end and a second strip end that is connected at the first strip end to a second side of the open end of the tube, wherein the second side of the open end of the tube is opposite from the first side of the open end of the tube; and
a flexible second triangular material having a third material end and a fourth material end, the flexible second triangular material being connected at the third material end to the second strip end of the flexible thin strip, wherein the fourth material end of the flexible second triangular material is wider than the third material end of the flexible second triangular material.

6. The device of claim 5, wherein:
the tube, the flexible first triangular material, the flexile second triangular material, and the flexible thin strip are molded as a single piece of material.

7. The device of claim 5 further comprising:
a first opening near the first corner of the flexible first triangular material;
a second opening near the second corner of the flexible first triangular material; and
a third opening near the fourth material end of the flexible second triangular material.

8. The device of claim 7 further comprising:
a belt having a first belt end and a second belt end, wherein the first belt end of the belt is attachable to the first opening and the second belt end of the belt is attachable to the second opening; and
an attachment strap having a first strap end and a second strap end, wherein the first strap end is attachable to the third opening and the second strap end is attachable to the belt.

9. The device of claim 7 further comprising:
a belt;
a first attachment strap attached to the belt that is attachable to the first opening;
a second attachment strap attached to the belt that is also attachable to the second opening;

10. A prophylactic device comprising:
an elongated hollow tube including a shaft, a rounded closed end, and an open end that forms an elongated hollow cavity within the tube, wherein the shaft of the tube maintains an elongated form regardless of whether an external object is inserted into the cavity of the tube;
a flexible first triangular material having a first material end and a second material end, the second material end having first and second corners, is the flexible first triangular material being connected at the first material end to a first side of the open end of the tube, wherein the second material end is wider than the first material end;
a flexible thin strip having a first strip end and a second strip end, the flexible thin strip being connected at the first strip end to a second side of the open end of the tube, wherein the second side of the open end of the tube is opposite from the first side of the open end of the tube; and
a flexible second triangular material having a third material end and a fourth material end, the flexible second triangular material being connected at the third material end to the second strip end of the flexible thin strip, wherein the fourth material end is wider than the third material end.
